(19) United States
(12) Patent Application Publication
(10) Pub. No.: US 2024/0257934 A1
(43) Pub. Date: Aug. 1, 2024

(54) MEDICATION ADMINISTRATION OF REMOTE SMART MEDICINE CABINATE

(71) Applicant: FAR EAST MEDICAL ELECTRONICS TECHNOLOGY CO., LTD., New Taipei (TW)

(72) Inventor: CHUN-NAN TENG, New Taipei (TW)

(21) Appl. No.: 18/414,712

(22) Filed: Jan. 17, 2024

(30) Foreign Application Priority Data
Jan. 18, 2023 (TW) 112102386

(51) Int. Cl. G16H 20/13; G16H 40/67

(52) U.S. Cl. CPC G16H 20/13; G16H 40/67

(57) ABSTRACT

A medicine administration of remote smart medicine cabinet, which allows patients to obtain a prescription through remote doctor diagnosis and treatment. The emergency rescue technician or medical professional activates the smart medicine cabinet based on the content of the prescription, and inputs the name of the prescribed medicine through the input unit. The controller of the smart medicine cabinet identifies the medicine based on the name of the medicine received. If the medicine is incorrect, the smart medicine cabinet issues a warning signal. If correct, the controller controls the matching medicine drawers to open, allowing the emergency rescue technician or medical professional to take out the required medicines. After re-confirming whether the medicine is consistent with the prescription, the medicine will be given to the patient to use, achieving the purpose of giving medicine after remote diagnosis and treatment.

This application claims the priority benefit of Taiwan patent application number 112102386, filed on Jan. 18, 2023.

### BACKGROUND OF THE INVENTION

### 1. Field of the Invention

The present invention provides a kind of medication administration of a remote smart medicine cabinet, especially a method of administering medicine through the smart medicine cabinet according to the prescription after remote diagnosis and treatment. Emergency technicians, medical professionals, etc. control the smart medicine cabinet and collect, identify, and confirm medicine according to the prescription issued by the doctor, and then hand it over to the patient, thereby achieving the purpose of correct medicine administration based on the patient's condition and remote diagnosis and treatment.

### 2. Description of the Related Art

Epidemics of infectious diseases are common around the world. In the early years, there was the severe acute respiratory syndrome (SARS) outbreak in 2003, and more recently, the new coronavirus pneumonia (COVID-19) broke out at the end of 2019. This disease, which is transmitted to new hosts through droplets and body fluids of the original host, is difficult to prevent. In particular, once this kind of 5 epidemic causes powerful complications with chronic diseases in the human body, its global fatality rate is rising, which is alarming. Although medical personnel such as doctors and nurses have the bounden duty to save lives, they spare no effort in treating epidemics. However, in addition to the responsibility of trying to treat patients, the medical system should also develop better medical equipment to prevent medical personnel from being infected by the disease during the treatment process and to provide effective and correct administration and medication to patients to alleviate their condition and recover.

As for cargo ships, cruise ships, or ocean-going fishing boats that travel on the sea for a long time, all kinds of ships sailing on the sea have many inconveniences in daily life because they are far away from the land. Moreover, once an illness occurs, effective treatment cannot be obtained because most ships are not equipped with medical personnel due to financial reasons. Once someone on the ship becomes ill, medical activities such as diagnosis, treatment, and medication cannot be carried out immediately. If the patients on the ship are mildly ill, they may be able to endure it until the ship docks before seeking medical treatment. However, those with more serious conditions sometimes have to be transported ashore by helicopter for treatment, which often causes the inconvenience of ships sailing on the sea for long periods of time.

Therefore, how to solve the current problems and troubles of people traveling on ships on the sea for long periods of time when they are sick, without immediate diagnosis and treatment, and medication, which causes the patients to endure pain on the ship and cannot seek medical treatment until the ship docks are the directions for improvement that relevant manufacturers in this industry are eager to study.

### SUMMARY OF THE INVENTION

In view of the above-mentioned problems and deficiencies, the inventor collected the relevant information, evaluated and considered it from many parties, and used his many years of experience accumulated in this industry to design, after various evaluations and considerations, he designed this medication administration of a remote smart medicine cabinet.

The main object of the present invention is that the medication administration of the remote smart medicine cabinet allows patients to obtain prescriptions through remote doctor diagnosis and treatment. The emergency rescue technician or medical professional starts the smart medicine cabinet based on the content of the prescription, and inputs at least one or more medicine names prescribed in the content of the prescription through the input unit. Then the controller of the smart medicine cabinet will identify the medicines based on the names of the medicines received. If the medicines are incorrect, the smart medicine cabinet will issue a warning signal. If it is correct, that is, the controller of the smart medicine cabinet controls at least one or more medicine drawers that match the medicine names to open, allowing the emergency rescue technician or medical professional to take out the required number of medicines that match the prescription content from the medicine drawers. After re-confirming whether the medicines are consistent with the prescription, the medicines will be given to the patient to use according to the doctor's instructions in the prescription, achieving the purpose of administering the medicine after remote diagnosis and treatment.

Another object of the present invention is that the patient can be diagnosed and treated by a remote doctor through remote video or phone calls, etc. The patient and doctor can conduct remote consultation via video or phone calls through the Global Satellite Positioning System (GPS), satellite network (Inmarsat), Internet or cloud system (Azure), etc., and then via fax, videos, photos or Internet transmission methods, etc., to obtain the prescription issued by the doctor. The prescription is transmitted by wireless transmission from the transmission unit set up in the remote medical center to the transceiver unit set up in the remote control center for the emergency rescue technician or medical professional to receive the prescription. The transmission unit of the remote medical center also performs wireless transmission with the transceiver unit of the control center through the Global Satellite Positioning System (GPS), Satellite Network (Inmarsat), Internet, or Cloud System (Azure). The content of the prescription can include the names of the medicines, dosage, days of use, method of use, side effects of the medicines, date and time of diagnosis and treatment, etc. It can also be used to indicate the name of the doctor, the name of the patient, the name of the patient's disease, medication instructions, precautions, doctor's instructions, and other related content. The remote medical center is further equipped with a storage unit, which can be used by the doctor to store information such as the prescription issued. The transmission unit can also receive medical-related information from the remote control center and transmit by the transceiver unit and store it in the storage unit.

Still another object of the present invention is that the smart medicine cabinet is provided with a control system. The control system is equipped with a controller, which can be a central processing unit, a chip or a microprocessor, etc., and can be electrically connected to an input unit, a start unit, a memory unit, a warning unit, a label machine, as well as multiple medicine drawers and a label machine, so that the emergency rescue technician or medical professional can activate the smart medicine cabinet through the start unit. The start unit is a keyboard, fingerprint reader, or facial recognition device installed on the smart medicine cabinet for entering passwords. As for the input unit of the smart medicine cabinet, it can be a plurality of keys or a touch screen, and it is electrically connected to the controller, allowing the emergency rescue technician or medical professional to input the relevant content and information of the prescription through the keys or the touch screen. The controller of the smart medicine cabinet identifies whether the input of at least one or more medicine names is correct based on the received at least one or more medicine names, and then compares it with the plural medicine names stored in the memory unit. The memory unit contains the name, quantity, usage, or efficacy of the medicines stored in the plural medicine drawers of the smart medicine cabinet. In addition, the warning unit electrically connected to the controller of the smart medicine cabinet is used to send warning signals. The warning unit can be a warning light, warning sound effect device (such as a buzzer, horn, or loudspeaker), etc., which can be used to issue warning signals such as warning lights or warning sounds. The smart medicine cabinet is electrically connected to the label machine through the controller, and the controller can control the label machine to output a label with the same content as the description.

Still, another object of the present invention is that the controller of the smart medicine cabinet is electrically connected to multiple medicine drawers respectively. The medicine drawers are used to store tablets, powders, capsules, internal medicines, injections, intravenous medicines, external medicines such as smearing medicines, ointments or patches, as well as bandages, cotton, cotton swabs, gauze, tapes, syringes, intravenous needles and other medical-related equipment, and various medical-related medicines.

### BRIEF DESCRIPTION OF THE DRAWINGS

FIG. 1 is a flow chart of the present invention (I).

FIG. 2 is a flow chart of the present invention (II).

FIG. 3 is a simple block diagram of the present invention.

FIG. 4 is a three-dimensional appearance view of the medicine cabinet of the present invention.

### DETAILED DESCRIPTION OF THE PREFERRED EMBODIMENT

In order to achieve the above objects and effect, the technical means and structure adopted in this invention, the drawings are hereby described in detail with respect to the preferred embodiments of this invention. Its features and functions are as follows for full understanding.

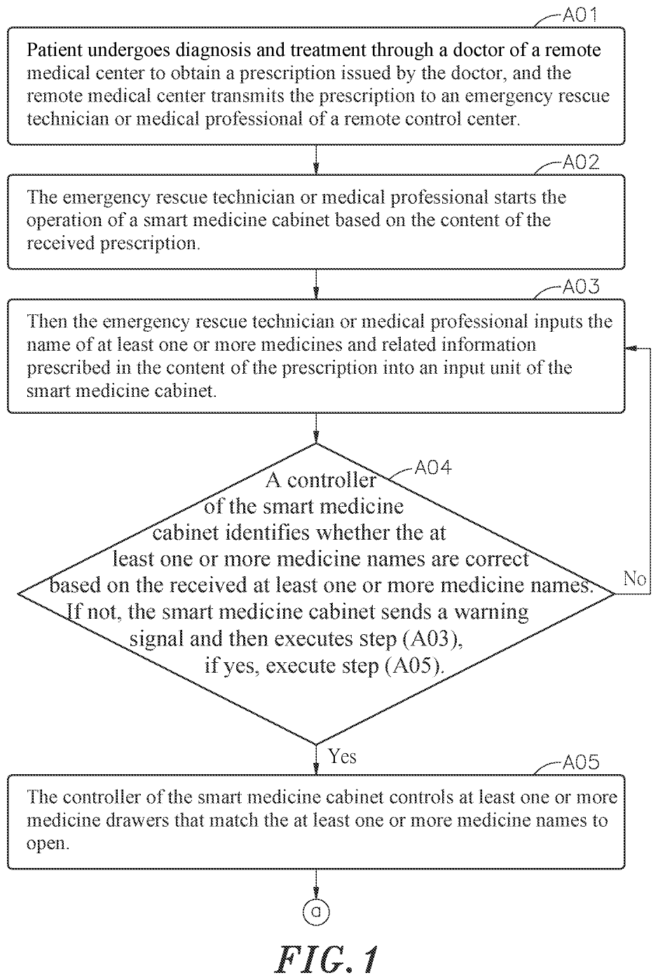

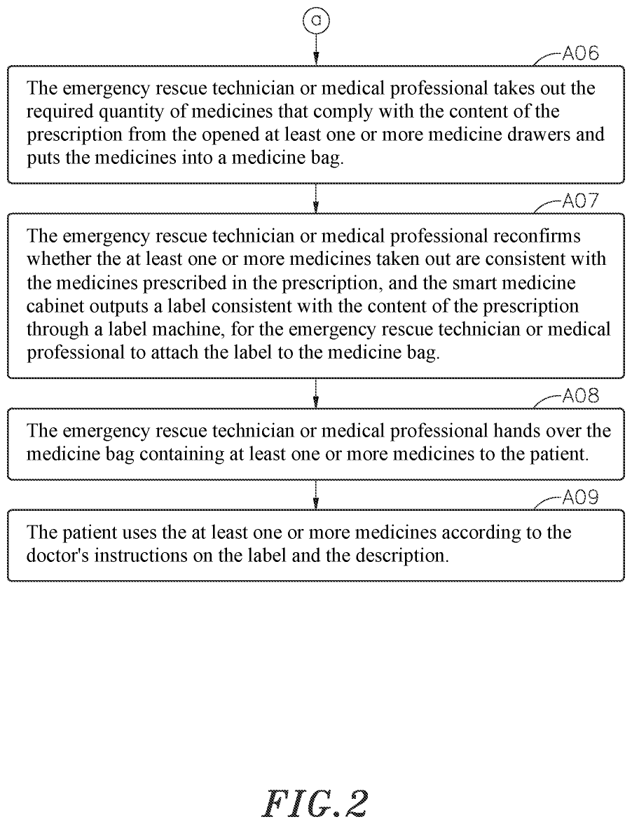

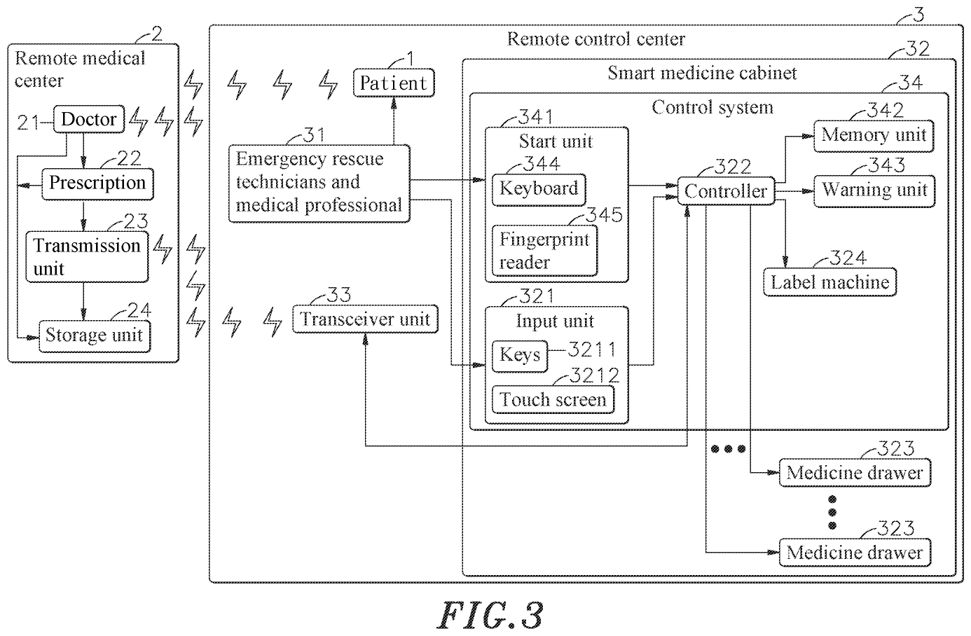

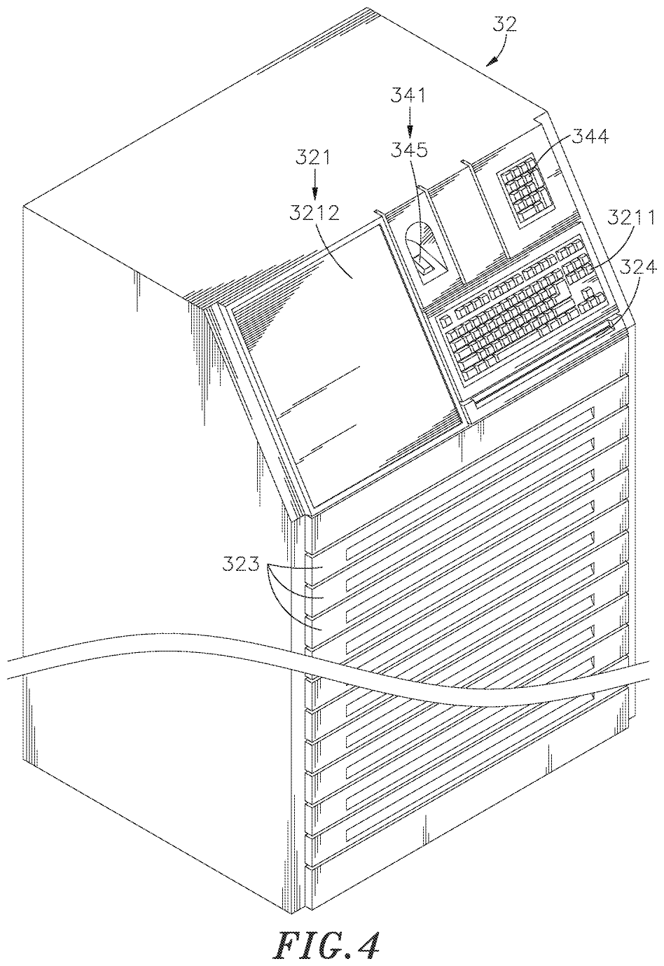

Please refer to FIGS. 1-4, which are respectively a flow chart of the present invention (I), a flow chart of the present invention (II), a simple block diagram of the present invention, and a three-dimensional appearance view of the medicine cabinet of the present invention. As the drawings clearly shown, the medication administration of remote smart medicine cabinet of the present invention can be implemented according to the following steps:

(A01) Patient 1 undergoes diagnosis and treatment through a doctor 21 of a remote medical center 2 to obtain a prescription 22 issued by the doctor 21, and the remote medical center 2 transmits the prescription 22 to an emergency rescue technician or medical professional 31 of a remote control center 3.

(A02) The emergency rescue technician or medical professional 31 starts the operation of a smart medicine cabinet 32 based on the content of the received prescription 22.

(A03) Then the emergency rescue technician or medical professional 31 inputs the name of at least one or more medicines and related information prescribed in the content of the prescription 22 into an input unit 321 of the smart medicine cabinet 32.

(A04) A controller 322 of the smart medicine cabinet 32 identifies whether the at least one or more medicine names are correct based on the received at least one or more medicine names. If not, the smart medicine cabinet 32 sends a warning signal and then executes Step (A03), if yes, execute step (A05).

(A05) The controller 322 of the smart medicine cabinet 32 controls at least one or more medicine drawers 323 that match the at least one or more medicine names to open.

(A06) The emergency rescue technician or medical professional 31 takes out the required quantity of medicines that comply with the content of the prescription 22 from the opened at least one or more medicine drawers 323 and puts the medicines into a medicine bag.

(A07) The emergency rescue technician or medical professional 31 reconfirms whether the at least one or more medicines taken out are consistent with the medicines prescribed in the prescription 22, and the smart medicine cabinet 32 outputs a label consistent with the content of the prescription 22 through a label machine 324, for the emergency rescue technician or medical professional 31 to attach the label to the medicine bag.

(A08) The emergency rescue technician or medical professional 31 hands over the medicine bag containing at least one or more medicines to the patient 1.

(A09) The patient 1 uses the at least one or more medicines according to the doctor's instructions on the label and the description.

The patient 1 in the above step (A01) is diagnosed and treated by the remote doctor 21 and can receive consultation, medical treatment, etc. through remote video or phone calls. The patient 1 and the doctor 21 can conduct remote video or phone calls through wireless transmission methods such as global satellite positioning system (GPS), Inmarsat satellite network, Internet, or cloud system (Azure). Then through wireless transmission methods such as encoding, decoding, synchronous and asynchronous network transmission, etc., to obtain the prescription 22 issued by the doctor 21. The description 22 is then transmitted to a transceiver unit 33 of the remote control center 3 by wireless transmission through a transmission unit 23 of the remote medical center 2 so that the emergency rescue technician or medical professional 31 can receive the prescription 22 through the transceiver unit 33. Wireless transmission between the transmission unit 23 of the remote medical center 2 and the transceiver unit 33 of the remote control center 3 can also be carried out through the global satellite system (GPS), Inmarsat satellite network, Internet or cloud system (Azure). The remote medical center 2 is further provided with a storage unit 24, which can be used by the doctor 21 to store information such as the prescription 22 issued.

The remote medical center 2 of the above step (A01) can be a hospital, medical center, clinic, or any medical institution on the remote land. According to the pain condition of patient 1 in the remote control center 3, remote diagnosis and treatment methods such as video or phone calls are carried out through the relevant department doctor 21 in the remote medical center 2. As for the remote control center 3, it can be installed on any of various ships such as cargo ships, cruise ships, or ocean-going fishing vessels sailing on the sea, or in a prison inmate control area. The remote control center 3 is equipped with emergency rescue technicians and medical professionals 31 such as medical-related nurses, nutritionists, pharmacists, or pharmacists. and a smart medicine cabinet 32 for storing medical-related drugs and equipment.

The smart medicine cabinet 32 in the above steps (A03) and (A04) is equipped with a control system 34, and the control system 34 is equipped with the controller 322. The controller 322 can be a central processing unit, a chip, or a microprocessor, and can be electrically connected to the input unit 321, a start unit 341, a memory unit 342 or a warning unit 343, as well as the multiple medicine drawers 323 and the label machine 324, so that the emergency rescue technician or medical professional 31 can activate the smart medicine cabinet 32 through the start unit 341. The start unit 341 is installed on the keyboard 344, fingerprint reader 345 or face recognition device on the smart medicine cabinet 32. The emergency rescue technician or medical professional 31 can start the start unit 341 by entering the password or authentication number (ID) through the keyboard 344, or using the fingerprint reader 345 for fingerprint recognition, or using the face recognition device for face recognition. The input unit 321 of the smart medicine cabinet 32 can be a plurality of keys 3211 or a touch screen 3212 and can be electrically connected to the controller 322. The emergency rescue technician or medical professional 31 can use the plural keys 3211 or the touch screen 3212 of the input unit 321 to input the relevant content and information of the prescription 22. The controller 322 of the smart medicine cabinet 32 can then control the electrically connected label machine 324 (which can be a printer or a label printer, etc.) to print a label with the same content as the description 22.

In addition, the transceiver unit 33 receives the prescription 22 and sends it to the memory unit 342 for storage. The emergency rescue technician or medical professional 31 can read the information content of the description 22 or other medical related information in the memory unit 342 through the controller 322 by using the keys 3211 or the touch screen 3212 of the input unit 321 of the control system 34. After the patient 1 has taken at least one or more medicines, the emergency rescue technician or medical professional 31 stores the medicine collection record in the memory unit 342 through the input unit 321 and the controller 322, and the controller 322 controls the transceiver unit 33 to transmit the data stored in the memory unit 342 to the transmission unit 23 of the remote medical center 2 through wireless transmission methods such as encoding, decoding, synchronous and asynchronous network transmission. The transmission unit 23 receives the medical and medication-related information transmitted from the transceiver unit 33 of the remote control center 3, and then stores it in the storage unit 24 so that the doctor 21 can use the patient 1's medicine collection situation, medical and medication-related information received from the memory unit 342 and recorded in the storage unit 24 to effectively control and interpret the information content.

The label content printed by the prescription 22 and the label machine 324 can include the name of the medicine, dosage, days of use, method of use, side effects of the medicine, or date and time of diagnosis and treatment, etc. It can also be used to mark doctor 21's name, patient's name, patient's disease name, medication information or precautions, or doctor's instructions, etc. Thus, the emergency rescue technician or medical professional 31 and the patient 1 can learn medication-related information through the prescription 22 or the labels.

In the above step (A05), the controller 322 of the smart medicine cabinet 32 identifies whether the input of at least one or more medicine names is correct based on the received at least one or more medicine names, and then compares it with the plurality of medicine names stored in the memory unit 342. The memory unit 342 contains the names, quantities, usage methods, efficacy, or side effects of the medicines stored in the plural medicine drawers 323 of the smart medicine cabinet 32. In addition, the warning unit 343 electrically connected to the controller 322 of the smart medicine cabinet 32 is used to send out a warning signal, and the warning unit 343 can be a warning light, a flashing light or a warning sound effect device (such as a buzzer, horn or loudspeaker), etc., which can be used to issue warning signals such as warning lights or warning sounds.

Furthermore, the controller 322 of the smart medicine cabinet 32 in the above steps (A04), (A05), and (A06) is electrically connected to the plurality of medicine drawers 323 respectively, and can control the opening or closing of the plurality of medicine drawers 323 (The controller 322 controls the opening and closing of multiple medicine drawers 323, which is a control method for general electronic systems, mechanisms, etc., and is not disclosed in detail because it is not a necessary technical feature of the present invention). The plurality of medicine drawers 323 can respectively store medical-related medicines such as tablets, powders, capsules, oral medicines, injections or intravenous medicines, or external medicines such as smearing medicines, ointments or patches, as well as bandages, cotton, cotton swabs, gauze, tape or syringes, intravenous needles and other medical-related equipment. The plurality of medicine drawers 323 allow the emergency rescue technicians or medical professionals 31 to take out the required medicines from the plurality of medicine drawers 323 according to the content of the description 22, and can put at least one or more medicines taken out into the medicine bag.

The smart medicine cabinet 32 in the above steps (A05) and (A07) can be electrically connected to the label machine 324 through the controller 322. Then the controller 322 can control the label machine 324 to output at least one or more labels with the same content as the description 22 for the emergency rescue technician or medical professional 31 to attach the label printed by the label machine 324 to the at least one or more medicines or medicine bags for the emergency rescue technician or medical professional 31 and the patient 1 to understand the information related to medication through the content marked on the prescription 22 or labels. Then the patient 1's medicine collection situation is stored in the memory unit 342 through the input unit 321 and the controller 322. Medicine-related or other information such as medicine preparation, replenishment, and medicine retrieval in the smart medicine cabinet 32 can also be stored in the memory unit 342.

In the above steps (A08) and (A09), the emergency rescue technician or medical professional 31 delivers the at least one or more medicines to the patient 1, so that the patient 1 can use the at least one or more medicines according to the doctor's instructions in the prescription 22 to relieve and cure the illness.

The above-mentioned patients 1 on various ships sailing on the sea, such as cargo ships, cruise ships, or ocean-going fishing vessels, can use the remote control center 3 installed on the ship to conduct video or phone calls with the doctor 21 of the remote medical center 2, and then the doctor 21 can issue a prescription 22. The emergency rescue technician or medical professional 31 of the remote control center 3 can obtain the required medicine from the smart medicine cabinet 32 according to the prescription 22 issued by the doctor 21. When any personnel on the ship (such as crew, management personnel, staff or tourists, etc.) is in pain, which we call the patient 1, can receive immediate medical diagnosis and treatment to achieve the purpose of correctly administering the medicine according to the condition of the patient 1 after remote diagnosis and treatment by the doctor 21 of the remote medical center 2. It enables the condition of the patient 1 to be controlled, alleviated and recovered as soon as possible. It also enables people who have been on various ships for a long time to receive proper medical care.

The above description is only a preferred embodiment of the present invention and does not limit the patent scope of the present invention. Therefore, all simple modifications and equivalent structural changes made by using the content of the specification and drawings of the present invention should be included in the patent scope of the present invention.

To sum up, the medication administration of the remote smart medicine cabinet of the present invention can really achieve its effect and purpose when used. Therefore, the present invention is sincerely an invention with excellent practicability. In order to meet the requirements for application for a new patent, I would like to file an application in accordance with the law. I hope that the review committee will approve this case as soon as possible to protect the hard work of the inventor. If the review committee has any doubts, please don't hesitate to send us a letter to instruct, the inventor will try his best to cooperate, thank you for your help.

What the invention claimed is:

1. A medication administration of remote smart medicine cabinet, comprising the steps of:
(A01) a patient undergoing diagnosis and treatment through a doctor of a remote medical center to obtain a prescription issued by said doctor, and said remote medical center transmitting said prescription to an emergency rescue technician or medical professional of a remote control center;
(A02) said emergency rescue technician or medical professional start the operation of a smart medicine cabinet based on the content of the received said prescription;
(A03) said emergency rescue technician or medical professional inputting the name of at least one medicine and related information prescribed in the content of said prescription into an input unit of said smart medicine cabinet;
(A04) a controller of said smart medicine cabinet identifying the correctness of the name of each of said at least one medicine based on the received name of each of said at least one medicine and then said smart medicine cabinet sending a warning signal and then executing Step (A03), if not, or executing step (A05), if yes;
(A05) said controller of said smart medicine cabinet controlling at least one medicine drawer of said smart medicine cabinet that matches the name of each of said at least one medicine to open;
(A06) said emergency rescue technician or medical professional taking out the required quantity of medicines that comply with the content of said prescription from the opened said at least one medicine drawer, and putting the medicines into a medicine bag;
(A07) said emergency rescue technician or medical professional reconfirming whether each of the said at least one medicine taken out is consistent with the respective medicine prescribed in said prescription, and said smart medicine cabinet outputting a label consistent with the content of said prescription through a label machine, for said emergency rescue technician or medical professional to attach the label to said medicine bag;
(A08) said emergency rescue technician or medical professional handing over said medicine bag containing said at least one medicine to said patient; and.
(A09) said patient using said at least one medicine according to the instructions of said doctor described in said label and said description.

2. The medication administration of remote smart medicine cabinet as claimed in claim 1, wherein in step (A01), said patient diagnosed and treated by said doctor through said remote medical center) is through remote video or phone calls, and then said prescription issued by said doctor is obtained through fax, video, photo or Internet transmission; said prescription is transmitted wirelessly from a transmission unit set up in said remote medical center to a transceiver unit set up in said remote control center for said emergency rescue technician or medical professional to receive said prescription and said remote medical center is further equipped with a storage unit for storing information content.

3. The medication administration of remote smart medicine cabinet as claimed in claim 2, wherein said patient and said doctor conduct remote video or phone consultation through the Global Satellite Positioning System (GPS), satellite network (Inmarsat), Internet or cloud system (Azure); said transmission unit of said remote medical center also uses the Global Satellite Positioning System (GPS), satellite network (Inmarsat), Internet or cloud system (Azure) to communicate with said transceiver unit of said remote control center wirelessly.

4. The medication administration of remote smart medicine cabinet as claimed in claim 1, wherein the content of said description in steps (A01) and (A03) is the name of the medicine, dosage, days of use, method of use, side effects of the medicine, and date and time of diagnosis and treatment, and said description also indicates the name of said doctor, the name of said patient, the disease name of said patient, medication instructions, precautions, and the instructions of said doctor for step (A09).

5. The medication administration of remote smart medicine cabinet as claimed in claim 1, wherein in steps (A03), (A04) and (A05) said smart medicine cabinet is equipped with a control system, said control system comprising said controller, a start unit, a memory unit, and a warning unit, said controller being selectively a central processing unit, a chip or a microprocessor electrically connected to said input unit, said start unit, said memory unit, said warning unit, said medicine drawers and said label machine for said emergency rescue technician or medical professional in step (A02) to start said smart medicine cabinet through said start unit, said start unit being selectively a keyboard, a fingerprint reader or a facial recognition device provided on said smart medicine cabinet for entering passwords; said input unit of said smart medicine cabinet in step (A03) is selectively a plurality of keys or a touch screen for inputting content and information related to said description.

6. The medication administration of remote smart medicine cabinet as claimed in claim 5, wherein in steps (A03) and (A04), said controller of said smart medicine cabinet identifies whether the input of the name of each of said at least one medicine is correct based on the received name of each of said at least one medicine and then compares the input with plural medicine names stored in said memory unit; said memory unit stores the name, quantity, usage and efficacy of the medicines stored in said medicine drawers of said smart medicine cabinet.

7. The medication administration of remote smart medicine cabinet as claimed in claim 5, wherein in step (A04), said smart medicine cabinet emits said warning signal, and said controller of said smart medicine cabinet is electrically connected to said warning unit to control said warning unit to emit said warning signal, said warning unit being selectively a warning light or a warning sound effect device (such as a buzzer, horn or loudspeaker), which emits said warning signal in the form of a warning light or warning voice.

8. The medication administration of remote smart medicine cabinet as claimed in claim 1, wherein in steps (A05) and (A06), said controller of said smart medicine cabinet is electrically connected to said medicine drawers respectively, and said medicine drawers are for storing tablets, powders, capsules, oral liquids, injections, intravenous medicines, external medicines for external application of medicines, ointments, patches, as well as bandages, cotton, cotton swabs, gauze, tapes, and medical related equipment including syringes and intravenous needles.

9. The medication administration of remote smart medicine cabinet as claimed in claim 1, wherein in steps (A05) and (A07), said smart medicine cabinet is electrically connected to said label machine through said controller, and said controller controls said label machine to output a label with the same content as said description.
